(19) United States
(12) Patent Application Publication
(10) Pub. No.: US 2018/0135073 A1
(43) Pub. Date: May 17, 2018

(54) CRISPR-BASED GENOME MODIFICATION AND REGULATION

(71) Applicant: SIGMA-ALDRICH CO. LLC, St. Louis, MO (US)

(72) Inventors: Fuqiang Chen, St. Louis, MO (US); Gregory D. Davis, St. Louis, MO (US)

(21) Appl. No.: 15/653,136

(22) Filed: Jul. 18, 2017

Related U.S. Application Data
(63) Continuation of application No. 14/213,895, filed on Mar. 14, 2014, now abandoned.
(60) Provisional application No. 61/794,422, filed on Mar. 15, 2013.

(51) Int. Cl. C12N 15/85; C12N 15/90; C12N 9/22

(52) U.S. Cl. CPC C12N 15/85; C07K 2319/09; C12N 9/22; C12N 15/902

(57) ABSTRACT

The present invention provides methods for modifying chromosomal sequences. In particular, methods are provided for using RNA-guided endonucleases or modified RNA-guided endonucleases to modify targeted chromosomal sequences.

### CROSS-REFERENCE TO RELATED APPLICATIONS

This application is a continuation application of U.S. patent application Ser. No. 14/213,895 filed on Mar. 14, 2014, which claims priority to U.S. Provisional Application Ser. No. 61/794,422, filed Mar. 15, 2013, each of which is hereby incorporated by reference in its entirety.

### FIELD OF THE INVENTION

The present disclosure relates targeted genome modification. In particular, the disclosure relates to methods of using RNA-guided endonucleases or modified versions thereof to modify targeted chromosomal sequences.

### BACKGROUND OF THE INVENTION

Targeted genome modification is a powerful tool for genetic manipulation of eukaryotic cells, embryos, and animals. For example, exogenous sequences can be integrated at targeted genomic locations and/or specific endogenous chromosomal sequences can be deleted, inactivated, or modified. Current methods rely on the use of engineered nuclease enzymes, such as, for example, zinc finger nucleases (ZFNs) or transcription activator-like effector nucleases (TALENs). These chimeric nucleases contain programmable, sequence-specific DNA-binding modules linked to a nonspecific DNA cleavage domain. Each new genomic target, however, requires the design of a new ZFN or TALEN comprising a novel sequence-specific DNA-binding module. Thus, these custom designed nucleases tend to be costly and time-consuming to prepare. Moreover, the specificities of ZFNs and TALENS are such that they can mediate off-target cleavages.

Thus, there is a need for a targeted genome modification technology that does not require the design of a new nuclease for each new targeted genomic location. Additionally, there is a need for a technology with increased specificity with few or no off-target effects.

### SUMMARY OF THE INVENTION

Among the various aspects of the present disclosure are methods for modifying chromosomal sequences using modified RNA-guided endonucleases. In particular, one method comprises introducing into a cell or embryo (a) two or more RNA-guided endonucleases or nucleic acid encoding two or more RNA-guided endonucleases and (b) two or more guiding RNAs or DNA encoding two or more guiding RNAs, wherein each guiding RNA guides one of the RNA-guided endonucleases to a targeted site in the chromosomal sequence and the RNA-guided endonuclease cleaves at least one strand of the chromosomal sequence at the targeted site. In some embodiments, each RNA-guided endonuclease is derived from a Cas9 protein and comprises at least two nuclease domains. In embodiments in which two RNA-guided endonucleases are introduced into the cell or embryo, each RNA-guided endonuclease is derived from a Cas9 protein and comprises at least two nuclease domains, wherein one of the nuclease domains of each of two RNA-guided endonucleases is modified such that each RNA-guided endonuclease cleaves one strand of a double-stranded sequence, and wherein the two RNA-guided endonucleases together introduce a double-stranded break in the chromosomal sequence that is repaired by a DNA repair process such that the chromosomal sequence is modified. In other embodiments, each RNA-guided endonuclease or Cas9-derived RNA-guided endonuclease introduces a double-stranded break in the chromosomal sequence that is repaired by a DNA repair process such that the chromosomal sequence is modified. In further embodiments, the method further comprises introducing into the cell at least one donor polynucleotide, wherein the donor polynucleotide comprises at least one sequence having substantial sequence identity with sequence on one side of the targeted site in the chromosomal sequence. In certain embodiments, the donor polynucleotide further comprises a donor sequence. In various embodiments, the cell is a human cell, a non-human mammalian cell, a stem cell, a non-mammalian vertebrate cell, an invertebrate cell, a plant cell, or a single cell eukaryotic organism. In other embodiments, the embryo is a non-human one cell embryo. In some embodiments, each RNA-guided endonuclease further comprises at least one additional domain chosen from a nuclear localization signal, a cell-penetrating domain, or a marker domain.

Other aspects and features of the disclosure are detailed below.

### BRIEF DESCRIPTION OF THE DRAWINGS

FIG. 1A diagrams genome modification using protein dimers in which a double stranded break created by a dimer composed of two fusion proteins, each of which comprises a Cas-like protein for DNA binding and a FokI cleavage domain. FIG. 1B depicts a double stranded break created by a dimer composed of a fusion protein comprising a Cas-like protein and a FokI cleavage domain and a zinc finger nuclease comprising a zinc finger (ZF) DNA-binding domain and a FokI cleavage domain.

FIG. 2A illustrates regulation of gene expression using RNA-guided fusion proteins comprising a Cas-like protein used for DNA binding and an “A/R” domain that activates or represses gene expression. FIG. 2B diagrams a fusion protein comprising a Cas-like protein for DNA binding and a epigenetic modification domain (“Epi-mod’) that affects epigenetic states by covalent modification of proximal DNA or proteins.

FIG. 3A diagrams a double stranded break created by two RNA-guided endonucleases that have been converted into nickases. FIG. 3B depicts two double stranded breaks created by two RNA-guided endonuclease having endonuclease activity.

### DETAILED DESCRIPTION OF THE INVENTION

The present disclosure provides RNA-guided DNA-binding fusion proteins. The fusion proteins comprise CRISPR/Cas-like proteins or fragments thereof and effector domains. Suitable effector domains include, without limit, cleavage domains, transcriptional activation domains, transcriptional repressor domains, epigenetic modification domains, as well as other domains discussed herein. Each fusion protein is guided to a specific chromosomal sequence by a specific guiding RNA, wherein the effector domain mediates targeted genome modification or gene regulation. In one aspect, the fusion proteins can function as dimers thereby increasing the length of the target site and increasing the likelihood of its uniqueness in the genome (thus, reducing off target effects). For example, endogenous CRISPR systems modify genomic locations based on DNA binding word lengths of approximately 14-15 bp (Cong et al., Science, 2103, 339:819-823). At this word size, only 5-7% of the target sites are unique within the genome (Iseli et al., PLos One, 2007, 2(6):e579). In contrast, DNA binding word sizes for zinc finger nucleases typically range from 30-36 bp, resulting in target sites that are approximately 85-87% unique within the human genome. The smaller sized DNA binding sites utilized by CRISPR systems limits and complicates design of targeted CRISP-based nucleases near desired locations, such as disease SNPs, small exons, start codons, and stop codons, as well as other locations within complex genomes. The present disclosure not only provides means for expanding the CRISPR DNA binding word length (i.e., so as to limit off-target activity), but further provides CRISPR fusion proteins having modified functionality. According, the disclosed CRISPR fusion proteins have increased target specificity and unique functionality(ies).

### (I) Fusion Proteins

One aspect of the present disclosure provides a fusion protein comprising a CRISPR/Cas-like protein or fragment thereof and an effector domain. The CRISPR/Cas-like protein is derived from a clustered regularly interspersed short palindromic repeats (CRISPR)/CRISPR-associated (Cas) system protein. The effector domain can be a cleavage domain, a transcriptional activation domain, a transcriptional repressor domain, or an epigenetic modification domain. The effector domain can also be a marker domain, such as reporter protein, e.g., GFP, horseradish peroxidase, and others known in the art.

(a) CRISPR/Cas-Like Protein

The fusion protein comprises a CRISPR/Cas-like protein or a fragment thereof. The CRISPR/Cas-like protein can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the CRISPR/Cas-like protein of the fusion protein is derived from a type II CRISPR/Cas system. In exemplary embodiments, the CRISPR/Cas-like protein of the fusion protein is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition and/or RNA binding domain. RNA recognition and/or RNA binding domains interact with the guiding RNA. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein of the fusion protein can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, nuclease (i.e., DNase, RNase) domains of the CRISPR/Cas protein can be modified, deleted, or inactivated. Alternatively, the CRISPR/Cas protein can be truncated to remove domains that are not essential for the function of the fusion protein. The CRISPR/Cas protein can also be truncated or modified to optimize the activity of the effector domain of the fusion protein.

In some embodiments, the CRISPR/Cas-like protein of the fusion protein can be derived from a wild type Cas9 protein or fragment thereof. In other embodiments, the CRISPR/Cas-like protein of the fusion protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek et al., Science, 2013, 337: 816-821). In some embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a nickase. Likewise, a histidine to alanine (H840A) conversion in a HNH domain converts the Cas9-derived protein into a nickase.

In other embodiments, both of the RuvC-like nuclease domain and the HNH-like nuclease domain can be modified or eliminated such that the Cas9-derived protein is unable to nick or cleave double stranded nucleic acid. In still other embodiments, all nuclease domains of the Cas9-derived protein can be modified or eliminated such that the Cas9-derived protein lacks all nuclease activity.

In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art. In an exemplary embodiment, the CRISPR/Cas-like protein of the fusion protein is derived from a Cas9 protein in which all the nuclease domains have been inactivated or deleted.

(b) Effector Domain

The fusion protein also comprises an effector domain. The effector domain can be a cleavage domain, a transcriptional activation domain, a transcriptional repressor domain, or an epigenetic modification domain. The effector domain can also be a nuclear localization signal, cell-penetrating or translocation domain, or a marker domain. The effector domain can be located at the carboxy or the amino terminal end of the fusion protein.

(i) Cleavage Domain

In some embodiments, the effector domain is a cleavage domain. As used herein, a “cleavage domain” refers to a domain that cleaves DNA. The cleavage domain can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains.

In some embodiments, the cleavage domain can be derived from a type II-S endonuclease. Type II-S endonucleases cleave DNA at sites that are typically several base pairs away the recognition site and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. In exemplary embodiments, the cleavage domain of the fusion protein is a FokI cleavage domain or a derivative thereof.

In certain embodiments, the type II-S cleavage can be modified to facilitate dimerization of two different cleavage domains (each of which is attached to a CRISPR/Cas-like protein or fragment thereof). For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI cleavage domains are targets for modification. For example, modified cleavage domains of FokI that form obligate heterodimers include a pair in which a first modified cleavage domain includes mutations at amino acid positions 490 and 538 and a second modified cleavage domain that includes mutations at amino acid positions 486 and 499 (Miller et al., 2007, Nat. Biotechnol, 25:778-785; Szczpek et al., 2007, Nat. Biotechnol, 25:786-793). For example, the Glu (E) at position 490 can be changed to Lys (K) and the Ile (I) at position 538 can be changed to K in one domain (E490K, I538K), and the Gln (Q) at position 486 can be changed to E and the I at position 499 can be changed to Leu (L) in another cleavage domain (Q486E, I499L). In other embodiments, modified FokI cleavage domains can include three amino acid changes (Doyon et al. 2011, Nat. Methods, 8:74-81). For example, one modified FokI domain (which is termed ELD) can comprise Q486E, I499L, N496D mutations and the other modified FokI domain (which is termed KKR) can comprise E490K, I538K, H537R mutations.

In exemplary embodiments, the effector domain of the fusion protein is a FokI cleavage domain or a modified FokI cleavage domain.

In embodiments wherein the effector domain is a cleavage domain, the cas9 can be modified as discussed herein such that its endonuclease activity is eliminated. For example, the cas9 can be modified by mutating the RuvC and HNH domains such that they no longer possess nuclease activity.

(ii) Transcriptional Activation Domain

In other embodiments, the effector domain of the fusion protein can be a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In some embodiments, the transcriptional activation domain can be, without limit, a herpes simplex virus VP16 activation domain, VP64 (which is a tetrameric derivative of VP16), a NFκB p65 activation domain, p53 activation domains 1 and 2, a CREB (cAMP response element binding protein) activation domain, an E2A activation domain, and an NFAT (nuclear factor of activated T-cells) activation domain. In other embodiments, the transcriptional activation domain can be Gal4, Gcn4, MLL, Rtg3, Gln3, Oaf1, Pip2, Pdr1, Pdr3, Pho4, and Leu3. The transcriptional activation domain may be wild type, or it may be a modified version of the original transcriptional activation domain. In some embodiments, the effector domain of the fusion protein is a VP16 or VP64 transcriptional activation domain.

In embodiments wherein the effector domain is a cleavage domain, the cas9 can be modified as discussed herein such that its endonuclease activity is eliminated. For example, the cas9 can be modified by mutating the RuvC and HNH domains such that they no longer possess nuclease activity.

(iii) Transcriptional Repressor Domain

In still other embodiments, the effector domain of the fusion protein can be a transcriptional repressor domain. In general, a transcriptional repressor domain interacts with transcriptional control elements and/or transcriptional regulatory proteins (i.e., transcription factors, RNA polymerases, etc.) to decrease and/or terminate transcription of a gene. Non-limiting examples of suitable transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, and MeCP2.

In embodiments wherein the effector domain is a cleavage domain, the cas9 can be modified as discussed herein such that its endonuclease activity is eliminated. For example, the cas9 can be modified by mutating the RuvC and HNH domains such that they no longer possess nuclease activity.

(iv) Epigenetic Modification Domain

In alternate embodiments, the effector domain of the fusion protein can be an epigenetic modification domain. In general, epigenetic modification domains alter gene expression by modifying the histone structure and/or chromosomal structure. Suitable epigenetic modification domains include, without limit, histone acetyltransferase domains, histone deacetylase domains, histone methyltransferase domains, histone demethylase domains, DNA methyltransferase domains, and DNA demethylase domains.

In embodiments wherein the effector domain is a cleavage domain, the cas9 can be modified as discussed herein such that its endonuclease activity is eliminated. For example, the cas9 can be modified by mutating the RuvC and HNH domains such that they no longer possess nuclease activity.

(c) Additional Domains

In some embodiments, the fusion protein further comprises at least one additional domain. Non-limiting examples of suitable additional domains include nuclear localization signals (NLSs), cell-penetrating or translocation domains, and marker domains.

In certain embodiments, the fusion protein can comprise at least one nuclear localization signal. In general, an NLS comprises a stretch of basic amino acids. Nuclear localization signals are known in the art (see, e.g., Lange et al., J. Biol. Chem., 2007, 282:5101-5105). For example, in one embodiment, the NLS can be a monopartite sequence, such as PKKKRKV (SEQ ID NO:1) or PKKKRRV (SEQ ID NO:2). In another embodiment, the NLS can be a bipartite sequence. In still another embodiment, the NLS can be KRPAATKKAGQAKKKK (SEQ ID NO:3). The NLS can be located at the N-terminus, the C-terminal, or in an internal location of the fusion protein.

In some embodiments, the fusion protein can comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:4). In another embodiment, the cell-penetrating domain can be TLM (PLSSIFSRIGDPPKKKRKV; SEQ ID NO:5), a cell-penetrating peptide sequence derived from the human hepatitis B virus. In still another embodiment, the cell-penetrating domain can be MPG (GALFLGWLGAAGSTMGAPKKKRKV; SEQ ID NO:5 or GALFLGFLGAAGSTMGAWSQPKKKRKV; SEQ ID NO:6). In additional embodiments, the cell-penetrating domain can be Pep-1 (KETWWETWWTEWSQPKKKRKV; SEQ ID NO:7), VP22, a cell penetrating peptide from Herpes simplex virus, or a polyarginine peptide sequence. The cell-penetrating domain can be located at the N-terminus, the C-terminal, or in an internal location of the fusion protein.

In still other embodiments, the fusion protein can comprise at least one marker domain. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

### (II) Fusion Protein Dimers

The present disclosure also provides dimers comprising at least one fusion protein from section (I). The dimer can be a homodimer or a heterodimer. In some embodiments, the heterodimer comprises two different fusion proteins. In other embodiments, the heterodimer comprises one fusion protein and an additional protein.

In some embodiments, the dimer is a homodimer in which the two fusion protein monomers are identical with respect to the primary amino acid sequence. In one embodiment where the dimer is a homodimer, the cas9 proteins are modified such that their endonuclease activity is eliminated, i.e., such that they have no functional nuclease domains. In certain embodiments wherein the cas9 proteins are modified such that their endonuclease activity is eliminated, each fusion protein monomer comprises an identical Cas9 like protein and an identical cleavage domain. The cleavage domain can be any cleavage domain, such as any of the exemplary cleavage domains provided herein. In one specific embodiment, the cleavage domain is the FokI cleavage domain.

In other embodiments, the dimer is a heterodimer of two different fusion proteins. For example, the CRISPR/Cas-like protein of each fusion protein can be derived from a different CRISPR/Cas protein or from an orthologous CRISPR/Cas protein from a different bacterial species. For example, each fusion protein can comprise a Cas9-like protein, which Cas9-like protein is derived from a different bacterial species. In these embodiments, each fusion protein would recognize a different target site (i.e., specified by the protospacer and/or PAM sequence). For example, the guiding RNAs could position the heterodimer to different but closely adjacent sites such that their nuclease domains results in an effective double stranded break in the target DNA. The heterodimer can also have modified cas9 proteins with nicking activity such that the nicking locations are different.

Alternatively, two fusion proteins can have different effector domains. In embodiments in which the effector domain is a cleavage domain, each fusion protein can contain a different modified cleavage domain. For example, each fusion protein can contain a different modified FokI cleavage domain, as detailed above in section (I)(b)(i). In these embodiments, the cas-9 proteins can be modified such that their endonuclease activities are eliminated.

As will be appreciated by those skilled in the art, the two fusion proteins forming a heterodimer can differ in both the CRISPR/Cas-like protein domain and the effector domain.

In any of the above-described embodiments, the homodimer or heterodimer can comprise at least one additional domain chosen from nuclear localization signals (NLSs), cell-penetrating, translocation domains and marker domains. Examples of suitable additional domains are detailed above in section (I)(c).

In any of the above-described embodiments, one or both of the cas9 proteins can be modified such that its endonuclease activity is eliminated or modified.

In still alternate embodiments, the heterodimer comprises one fusion protein and an additional protein. For example, the additional protein can be a nuclease. In one embodiment, the nuclease is a zinc finger nuclease. A zinc finger nuclease comprises a zinc finger DNA binding domain and a cleavage domain. A zinc finger recognizes and binds three (3) nucleotides. A zinc finger DNA binding domain can comprise from about three zinc fingers to about seven zinc fingers. The zinc finger DNA binding domain can be derived from a naturally occurring protein or it can be engineered. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. The cleavage domain of the zinc finger nuclease can be any cleavage domain detailed above in section (I)(b)(i). In exemplary embodiments, the cleavage domain of the zinc finger nuclease is a FokI cleavage domain or a modified FokI cleavage domain. Such a zinc finger nuclease will dimerize with a fusion protein comprising a FokI cleavage domain or a modified FokI cleavage domain.

In some embodiments, the zinc finger nuclease can comprise at least one additional domain chosen from nuclear localization signals (NLSs), cell-penetrating or translocation domains. Examples of suitable additional domains are detailed above in section (I)(c).

### (III) Nucleic Acids Encoding Fusion Proteins

Another aspect of the present disclosure provides nucleic acids encoding any of the fusion proteins or protein dimers described above in sections (I) and (II). The nucleic acid encoding the fusion protein can be RNA or DNA. In one embodiment, the nucleic acid encoding the fusion protein is mRNA. In another embodiment, the nucleic acid encoding the fusion protein is DNA. The DNA encoding the fusion protein can be present in a vector (see below).

The nucleic acid encoding the fusion protein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware (e.g., OPTIMIZER or OptimumGene™). Commercial codon optimization programs are also available.

In some embodiments, DNA encoding the fusion protein can be operably linked to at least one promoter control sequence. In some iterations, the DNA coding sequence can be operably linked to a promoter control sequence for expression in the eukaryotic cell or animal of interest. The promoter control sequence can be constitutive or regulated. The promoter control sequence can be tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. The promoter sequence can be wild type or it can be modified for more efficient or efficacious expression. In one exemplary embodiment, the DNA encoding the fusion is operably linked to a CMV promoter for constitutive expression in mammalian cells.

In other embodiments, the sequence encoding the fusion protein can be operably linked to a promoter sequence that is recognized by a phage RNA polymerase for in vitro mRNA synthesis. For example, the promoter sequence can be a T7, T3, or SP6 promoter sequence or a variation of a T7, T3, or SP6 promoter sequence. In an exemplary embodiment, the DNA encoding the fusion protein is operably linked to a T7 promoter for in vitro mRNA synthesis using T7 RNA polymerase.

In alternate embodiments, the sequence encoding the fusion protein can be operably linked to a promoter sequence for in vitro expression of the fusion protein in bacterial or eukaryotic cells. In such embodiments, the expression fusion protein can be purified for use in the methods detailed below in section (IV). Suitable bacterial promoters include, without limit, T7 promoters, lac operon promoters, trp promoters, variations thereof, and combinations thereof. An exemplary bacterial promoter is tac which is a hybrid of trp and lac promoters. Non-limiting examples of suitable eukaryotic promoters are listed above.

In various embodiments, the DNA encoding the fusion protein can be present in a vector. Suitable vectors include plasmid vectors, phagemids, cosmids, artificial/mini-chromosomes, transposons, and viral vectors. In one embodiment, the DNA encoding the fusion protein is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

### (IV) Method for Using a Fusion Protein to Modify a Chromosomal Sequence or Regulate Expression of a Chromosomal Sequence

Another aspect of the present disclosure encompasses a method for modifying a chromosomal sequence or regulating expression of a chromosomal sequence in a cell, embryo, or animal. The method comprises introducing into the cell or embryo (a) at least one fusion protein or a nucleic acid encoding the fusion protein, the fusion protein comprising a CRISPR/Cas-like protein or a fragment thereof and an effector domain, and (b) at least one guiding RNA or DNA encoding the guiding RNA, wherein the guiding RNA guides the CRISPR/Cas-like protein of the fusion protein to a targeted site in the chromosomal sequence and the effector domain of the fusion protein modifies the chromosomal sequence or regulates expression of the chromosomal sequence. In some embodiments, the method further comprises introducing into the cell or embryo at least one donor polynucleotide comprising at least one sequence having substantial sequence identity with sequence on one side of the targeted site in the chromosomal sequence. In other embodiments, the method further comprises introducing into the cell or embryo at least one donor polynucleotide comprising sequence having substantial sequence identity with sequence on both sides of the targeted site in the chromosomal sequence. In embodiments in which the effector domain is a cleavage domain, the cas9 protein is modified such that the endonuclease activity is eliminated.

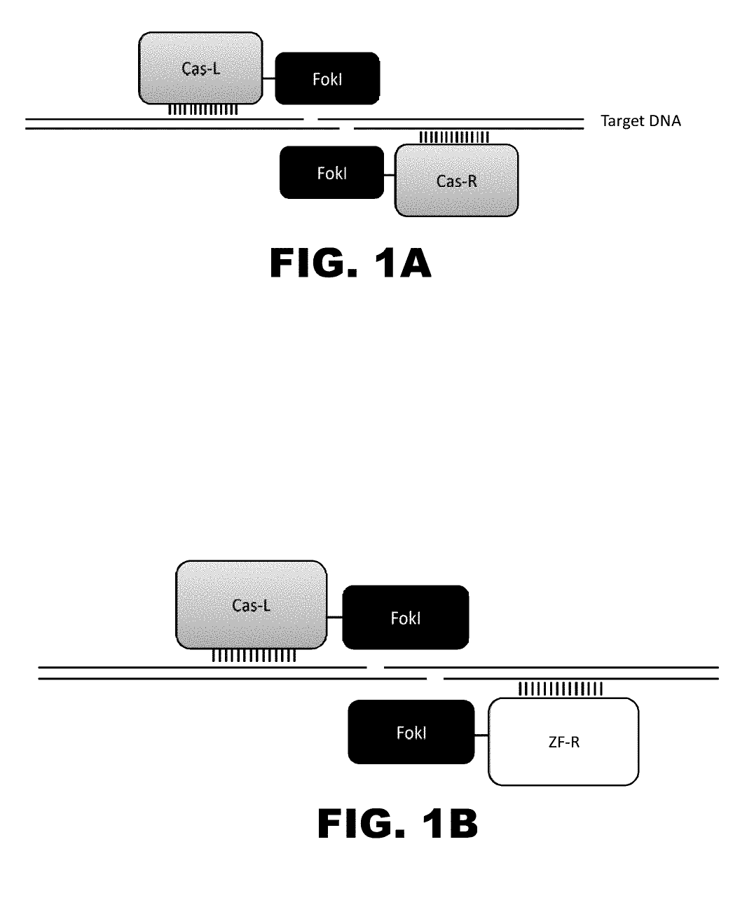

In certain embodiments in which the fusion protein comprises a cleavage domain (e.g., a FokI cleavage domain or a modified FokI cleavage domain), the method can comprise introducing into the cell or embryo one fusion protein (or nucleic acid encoding one fusion protein) and two guiding RNAs (or DNA encoding two guiding RNAs). The two guiding RNAs direct the fusion protein to two different chromosomal sequences, wherein the fusion protein dimerizes such that the two cleavage domains can introduce a double stranded break into the chromosomal sequence. The double-stranded break is repaired by a DNA repair process such that the chromosomal sequence is modified. See FIG. 1A.

In other embodiments in which the fusion protein comprises a cleavage domain (e.g., a FokI cleavage domain or a modified FokI cleavage domain), the method can comprise introducing into the cell or embryo two different fusion proteins (or nucleic acid encoding two different fusion proteins) and two guiding RNAs (or DNA encoding two guiding RNAs). The fusion proteins can differ as detailed above in section (II). Each guiding RNA directs a fusion protein to a specific chromosomal sequence, wherein the fusion proteins dimerize such that the two cleavage domains can introduce a double stranded break into the chromosomal sequence. The double-stranded break is repaired by a DNA repair process such that the chromosomal sequence is modified.

In another embodiment, the method can comprise introducing into the cell or embryo one fusion protein (or nucleic acid encoding one fusion protein), one guiding RNA (or DNA encoding one guiding RNA), and one zinc finger nuclease (or nucleic acid encoding the zinc finger nuclease). The guiding RNA directs the fusion protein to a specific chromosomal sequence, and the zinc finger nuclease is directed to another chromosomal sequence, wherein the fusion protein and the zinc finger nuclease dimerize such that the cleavage domain of the fusion protein and the cleavage domain of the zinc finger nuclease can introduce a double stranded break into the chromosomal sequence. The double-stranded break is repaired by a DNA repair process such that the chromosomal sequence is modified. See FIG. 1B.

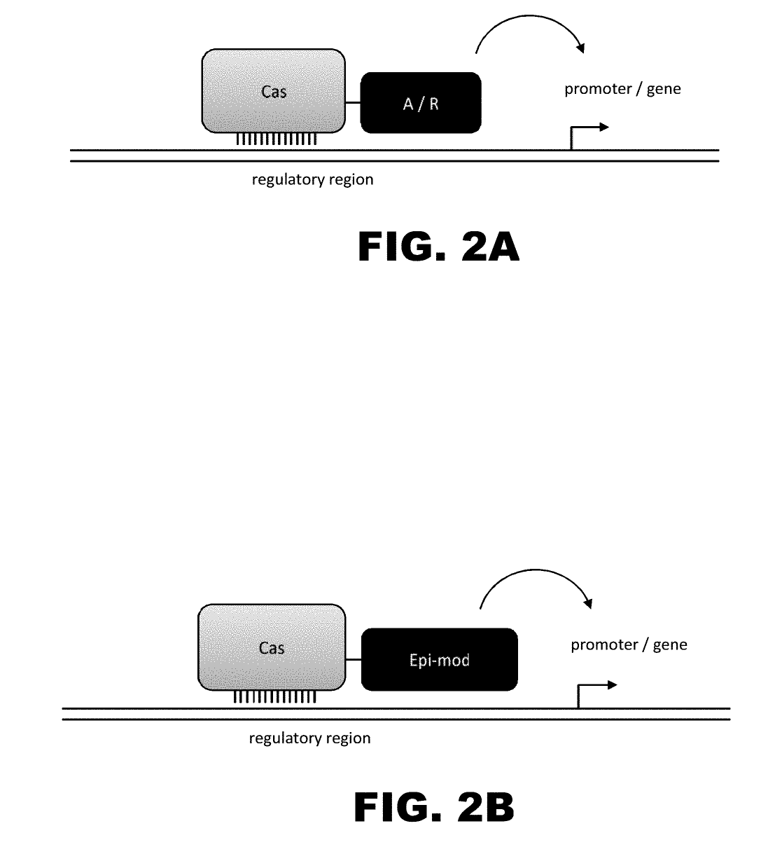

In still other embodiments in which the effector domain of the fusion protein is a transcriptional activation domain, a transcriptional repressor domain, or an epigenetic modification domain, the method can comprise introducing into the cell or embryo one fusion protein (or nucleic acid encoding one fusion protein) and one guiding RNA (or DNA encoding one guiding RNA). The guiding RNA directs the fusion protein to a specific chromosomal sequence, wherein the effector domain regulates expression of the chromosomal sequence. See FIG. 2A, 2B.

(a) Target Site

The fusion protein in conjunction with the guiding RNA is directed to a target site in the chromosomal sequence. The target site has no sequence limitation except that the sequence is immediately followed (downstream) by a consensus sequence. This consensus sequence is also known as a protospacer adjacent motif (PAM). Examples of PAM include, but are not limited to, NGG, NGGNG, and NNAGAAW (wherein N is defined as any nucleotide and W is defined as either A or T). The target site can be in the coding region of a gene, in an intron of a gene, in a control region between genes, etc. The gene can be a protein coding gene or an RNA coding gene.

(b) Fusion Protein

Fusion proteins and nucleic acids encoding fusion proteins are described above in sections (I)-(III). In some embodiments, the fusion protein or proteins can be introduced into the cell or embryo as an isolated protein. In one embodiment, the fusion protein can comprise at least one cell-penetrating domain, which facilitates cellular uptake of the protein. In other embodiments, an mRNA molecule or molecules encoding the fusion protein or proteins can be introduced into the cell or embryo. In still other embodiments, a DNA molecule or molecules encoding the fusion protein or proteins can be introduced into the cell or embryo. In general, DNA sequence encoding the fusion protein is operably linked to a promoter sequence that will function in the cell or embryo of interest. The DNA sequence can be linear, or the DNA sequence can be part of a vector. In still other embodiments, the fusion protein can be introduced into the cell or embryo as an RNA-protein complex comprising the fusion protein and the guiding RNA.

In alternate embodiments, DNA encoding the fusion protein can further comprise sequence encoding the guiding RNA. In general, the DNA sequence encoding the fusion protein and the guiding RNA is operably linked to appropriate promoter control sequences (such as the promoter control sequences discussed herein for fusion protein and guiding RNA expression) that allow the expression of the fusion protein and the guiding RNA, respectively, in the cell or embryo. The DNA sequence encoding the fusion protein and the guiding RNA can further comprise additional expression control, regulatory, and/or processing sequence(s). The DNA sequence encoding the fusion protein and the guiding RNA can be linear or can be part of a vector.

(c) Guiding RNA

A guiding RNA interacts with the CRISPR/Cas-like protein of the fusion protein to guide the fusion protein to a specific target site, wherein the effector domain of the fusion protein modifies the chromosomal sequence or regulates expression of the chromosomal sequence.

Each guiding RNA comprises three regions: a first region at the 5′ end that is complementary to the target site in the chromosomal sequence, a second internal region that forms a stem loop structure, and a third 3′ region that remains essentially single-stranded. The first region of each guiding RNA is different such that each guiding RNA guides a fusion protein to a specific target site. The second and third regions of each guiding RNA can be the same in all guiding RNAs.

The first region of the guiding RNA is complementary to the target site in the chromosomal sequence such that the first region of the guiding RNA can base pair with the target site. In various embodiments, the first region of the guiding RNA can comprise from about 10 nucleotides to more than about 25 nucleotides. For example, the region of base pairing between the first region of the guiding RNA and the target site in the chromosomal sequence can be about 10, 11, 12, 13, 14, 15, 16, 17, 18, 19, 20, 22, 23, 24, 25, or more than 25 nucleotides in length. In an exemplary embodiment, the first region of the guiding RNA is about 20 nucleotides in length.

The guiding RNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guiding RNA also comprises a third region at the 3′ end that remains essentially single-stranded. Thus, the third region has no complementarity to any chromosomal sequence in the cell of interest and has no complementarity to the rest of the guiding RNA. The length of the third region can vary. In general, the third region is more than about 4 nucleotides in length. For example, the length of the third region can range from about 5 to about 30 nucleotides in length.

In another embodiment, the guiding RNA can comprise two separate molecules. The first RNA molecule can comprise the first region of the guiding RNA and one half of the “stem” of the second region of the guiding RNA. The second RNA molecule can comprise the other half of the “stem” of the second region of the guiding RNA and the third region of the guiding RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence.

In embodiments in which the guiding RNA is introduced into the cell as a DNA molecule, the guiding RNA coding sequence can be operably linked to promoter control sequence for expression of the guiding RNA in the eukaryotic cell. For example, the RNA coding sequence can be operably linked to a promoter sequence that is recognized by RNA polymerase III (Pol III). Examples of suitable Pol III promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter.

The DNA molecule encoding the guiding RNA can be linear or circular. In some embodiments, the DNA sequence encoding the guiding RNA can be part of a vector. Suitable vectors include plasmid vectors, phagemids, cosmids, artificial/mini-chromosomes, transposons, and viral vectors. In an exemplary embodiment, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

(d) Optional Zinc Finger Nuclease

In some embodiments, the method comprises introducing into the cell or embryo a zinc finger nuclease or nucleic acid encoding the zinc finger nuclease. Zinc finger nucleases are described above in section (II). In some embodiments, the zinc finger nuclease can be introduced into the cell or embryo as an isolated protein. In one embodiment, the zinc finger nuclease can comprise at least one cell-penetrating domain, which facilitates cellular uptake of the protein. In other embodiments, an mRNA molecule encoding the zinc finger nuclease can be introduced into the cell or embryo. In other embodiments, a DNA molecule encoding the zinc finger nuclease can be introduced into the cell or embryo. In general, the DNA sequence encoding the zinc finger nuclease is operably linked to a promoter sequence that will function in the cell or embryo of interest. The DNA sequence can be linear, or the DNA sequence can be part of a vector.

(e) Optional Donor Polynucleotide

The method optionally also comprises introducing into the cell or embryo at least one donor polynucleotide comprising at least one sequence having substantial sequence identity with sequence on one side of the targeted site in the chromosomal sequence. As detailed below, the donor polynucleotide can comprise additional sequence elements.

The donor polynucleotide generally comprises a donor sequence. The donor sequence can be an exogenous sequence. As used herein, an “exogenous” sequence refers to a sequence that is not native to the cell or embryo, or a chromosomal sequence whose native location in the genome of the cell or embryo is in a different chromosomal location. For example, the donor sequence can comprise a protein coding gene, which can be operably linked to an exogenous promoter control sequence such that, upon integration into the cell or embryo, the cell or embryo expresses the protein coded by the integrated gene. Alternatively, the exogenous sequence can be integrated into the chromosomal sequence such that its expression is regulated by an endogenous promoter control sequence. Integration of an exogenous gene into the chromosomal sequence is termed a “knock in.” In other embodiments, the exogenous sequence can be a transcriptional control sequence, another expression control sequence, an RNA coding sequence, and so forth.

In some embodiments, the donor sequence of the donor polynucleotide can be a sequence that is essentially identical to a portion of the chromosomal sequence at or near the targeted site, but which comprises at least one nucleotide change. Thus, the donor sequence can comprise a modified version of the wild type sequence at the targeted site such that, upon integration or exchange with the chromosomal sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the integration of the modified sequence, the cell or embryo can produce a modified gene product from the targeted chromosomal sequence.

As can be appreciated by those skilled in the art, the length of the donor sequence can and will vary. For example, the donor sequence can vary in length from several nucleotides to hundreds of nucleotides to hundreds of thousands of nucleotides.

In some embodiments, the donor sequence in the donor polynucleotide is flanked by an upstream sequence and a downstream sequence, which have substantial sequence identity to sequences located upstream and downstream, respectively, of the targeted site in the chromosomal sequence. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the targeted site. Similarly, the downstream sequence refers to a nucleic acid sequence that shares substantial sequence identity with a chromosomal sequence downstream of the targeted site. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the targeted site. In an exemplary embodiment, the upstream and downstream sequences in the donor polynucleotide can have about 95% or 100% sequence identity with chromosomal sequences upstream or downstream to the targeted site. In one embodiment, the upstream sequence shares substantial sequence identity with a chromosomal sequence located immediately upstream of the targeted site (i.e., adjacent to the targeted site). In other embodiments, the upstream sequence shares substantial sequence identity with a chromosomal sequence that is located within about one hundred (100) nucleotides upstream from the targeted site. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the targeted site. In one embodiment, the downstream sequence shares substantial sequence identity with a chromosomal sequence located immediately downstream of the targeted site (i.e., adjacent to the targeted site). In other embodiments, the downstream sequence shares substantial sequence identity with a chromosomal sequence that is located within about one hundred (100) nucleotides downstream from the targeted site. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the targeted site.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In exemplary embodiments, upstream and downstream sequences can range in length from about 500 to about 1500 nucleotides.

Donor polynucleotides comprising the upstream and downstream sequences with sequence similarity to the targeted chromosomal sequence can be linear or circular. In embodiments in which the donor polynucleotide is circular, it can be part of a vector (detailed above). For example, the vector can be a plasmid vector.

(f) Introducing into the Cell or Embryo

The fusion protein(s) and/or zinc finger protein (or nucleic acid(s) encoding the fusion protein(s) and/or zinc finger protein), the guiding RNA(s) or DNAs encoding the guiding RNAs, and the optional donor polynucleotide(s) can be introduced into a cell or embryo by a variety of means. Typically, the embryo is a fertilized one-cell stage embryo of the species of interest. In some embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001). In other embodiments, the molecules are introduced into the cell or embryo by microinjection. For example, the molecules can be injected into the pronuclei of one cell embryos.

The fusion protein(s) and/or zinc finger protein (or nucleic acid(s) encoding the fusion protein(s) and/or zinc finger protein), the guiding RNA(s) or DNAs encoding the guiding RNAs, and the optional donor polynucleotide(s) can be introduced into the cell or embryo simultaneously or sequentially. The ratio of the fusion protein (or its encoding nucleic acid) to the guiding RNA(s) (or DNAs encoding the guiding RNA), generally will be approximately stoichiometric such that they can form an RNA-protein complex. Similarly, the ratio of two different fusion proteins (or encoding nucleic acids) will be approximately stoichiometric, as will the ratio of fusion protein to zinc finger nuclease (or encoding nucleic acids). In one embodiment, the fusion protein and the guiding RNA(s) (or the DNA sequence encoding the fusion protein and the guiding RNA(s)) are delivered together within the same nucleic acid or vector.

(g) Culturing the Cell or Embryo

The method further comprises maintaining the cell or embryo under appropriate conditions such that the guiding RNA guides the fusion protein to the targeted site in the chromosomal sequence, and the effector domain of the fusion protein modifies the chromosomal sequence or regulates expression of the chromosomal sequence.

In embodiments in which the fusion protein comprises a cleavage domain and no donor polynucleotide is introduced into the cell or embryo, the double-stranded break introduced by the fusion protein can be repaired via a non-homologous end-joining (NHEJ) repair process. Because NHEJ is error-prone, deletions of at least one nucleotide, insertions of at least one nucleotide, substitutions of at least one nucleotide, or combinations thereof can occur during the repair of the break. Accordingly, the sequence at the chromosomal sequence can be modified such that the reading frame of a coding region can be shifted and that the chromosomal sequence is inactivated or “knocked out.” An inactivated protein-coding chromosomal sequence does not give rise to the protein coded by the wild type chromosomal sequence.

In embodiments in which the fusion protein comprises a cleavage domain and a donor polynucleotide comprising upstream and downstream sequences is introduced into the cell or embryo, the double-stranded break introduced by the fusion protein can be repaired by a homology-directed repair (HDR) process such that the donor sequence is integrated into the chromosomal sequence. As detailed above in section (II)(c), an exogenous sequence can be integrated into the genome of the cell or the targeted chromosomal sequence can be modified by exchange of a modified sequence for the wild type chromosomal sequence.

In embodiments in which the effector domain of the fusion protein comprises a transcriptional activation domain, a transcriptional repressor domain, or an epigenetic modification domain, the effector domain modulates gene expression at the targeted chromosomal sequence.

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the RNA endonuclease and guiding RNA, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal derived from the embryo. Such an animal would comprise the modified chromosomal sequence in every cell of the body.

(h) Cell and Embryo Types

A variety of eukaryotic cells are suitable for use in the method. In various embodiments, the cell can be a human cell, a non-human mammalian cell, a non-mammalian vertebrate cell, an invertebrate cell, an insect cell, a plant cell, a yeast cell, or a single cell eukaryotic organism. A variety of embryos are suitable for use in the method. For example, the embryo can be a one cell non-human mammalian embryo. Exemplary mammalian embryos, including one cell embryos, include without limit mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell or the embryo is a mammalian embryo.

Non-limiting examples of suitable mammalian cells include Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells. An extensive list of mammalian cell lines may be found in the American Type Culture Collection catalog (ATCC, Manassas, Va.).

### (V) Method for Modifying a Chromosomal Sequence Using Modified RNA-Guided Endonucleases

Yet another aspect of the present disclosure encompasses a method for modifying a chromosomal sequence in a cell, embryo, or animal. The method comprises introducing into the cell or embryo (a) two or more RNA-guided endonucleases or nucleic acid encoding two or more RNA-guided endonucleases and (b) two or more guiding RNAs or DNA encoding two or more guiding RNAs, wherein each guiding RNA guides one of the RNA-guided endonucleases to a targeted site in the chromosomal sequence and the RNA-guided endonuclease cleaves at least one strand of the chromosomal sequence at the targeted site. In some embodiments, the method further comprises introducing into the cell or embryo at least one donor polynucleotide comprising at least one sequence having substantial sequence identity with sequence on one side of the targeted site in the chromosomal sequence.

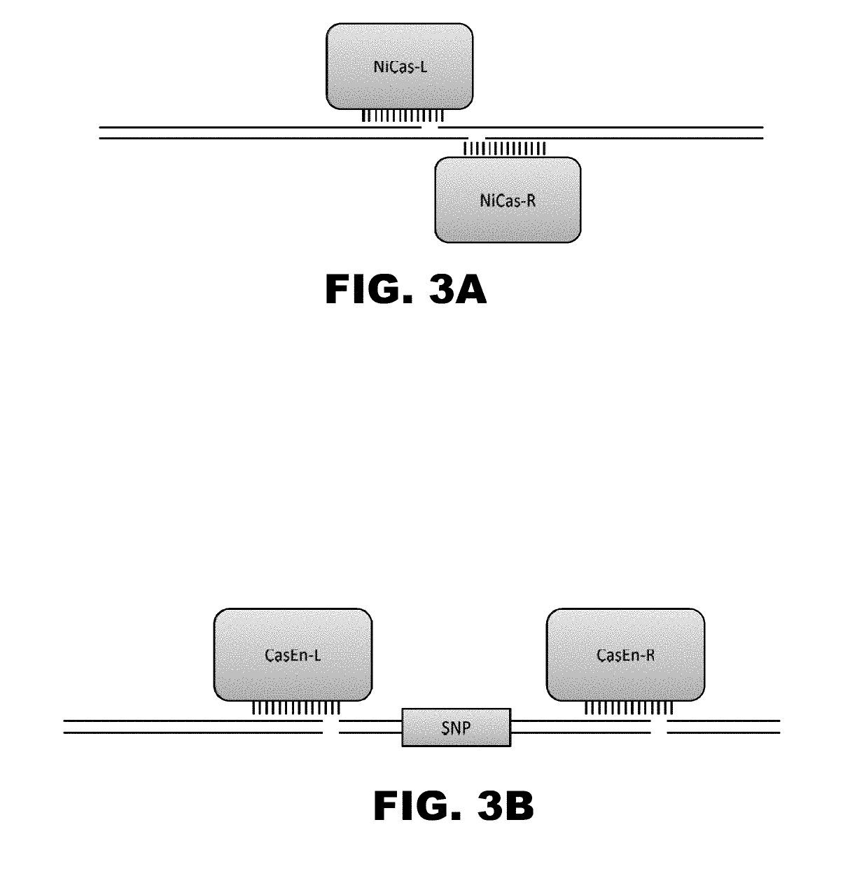

In one embodiment, the method comprises introducing two RNA-guided endonucleases that each has been modified to cleave one strand of a double-stranded sequence. Thus, the two RNA-guided endonucleases together introduce a double-stranded break in the chromosomal sequence. The two RNA-guided endonucleases can be directed by their respective guiding RNAs to the same, nearby (i.e., different but adjacent or close), or different target locations. The double-stranded break is repaired by a DNA repair process such that the chromosomal sequence is modified. See FIG. 3A. In embodiments in which no donor polynucleotide is introduced into the cell or embryo, the double-stranded break can be repaired via an error-prone, non-homologous end-joining (NHEJ) repair process. In embodiments in which a donor polynucleotide is introduced into the cell or embryo, the double-stranded break can be repaired by a homology-directed repair (HDR) process such that donor sequence in the donor polynucleotide can be integrated into or exchanged with the chromosomal sequence.

In another embodiment, the method comprises introducing two RNA-guided endonucleases, each of which introduces a double-stranded break in the chromosomal sequence. The two RNA-guided endonucleases can be directed by their respective guiding RNAs to nearby (i.e., different but adjacent or close) or different target locations. The double-stranded breaks are repaired by a DNA repair process such that the chromosomal sequence is modified. For example, the sequence between the two double-stranded breaks can be deleted, thereby modifying the chromosomal sequence. Alternatively, an optional donor polynucleotide comprising a donor sequence could have been also introduced into the cell or embryo. During repair of the double-stranded breaks, the donor sequence in the donor polynucleotide can be exchanged with the sequence between the two double-stranded breaks, thereby modifying the chromosomal sequence. See FIG. 3B.

The RNA-guided endonuclease can be derived from any of the CRISPR-Cas-like proteins detailed above in section (I). In exemplary embodiments, the RNA-guided endonuclease is derived from a Cas9 protein. In some embodiments, the Cas9-derived RNA-guided endonuclease comprises two functional nuclease domains. In other embodiments, at least one of the nuclease domains of the Cas9-derived RNA-guided endonuclease can be modified as detailed above in section (I) such that the Cas9-derived RNA-guided endonuclease cleaves one strand of a double stranded nucleic acid sequence.

In some embodiments, the RNA-guided endonuclease can be introduced into the cell as an isolated protein. In other embodiments, an mRNA molecule encoding the RNA-guided endonuclease can be introduced into the cell or embryo. In still other embodiments, a DNA molecule encoding the RNA-guided endonuclease can be introduced into the cell or embryo. In general, the DNA sequence encoding the RNA-guided endonuclease is operably linked to a promoter sequence that will function in the cell or embryo of interest. The DNA sequence can be linear, or the DNA sequence can be part of a vector. In alternate embodiments, the RNA-guided endonuclease can be introduced into the cell as a RNA-protein complex comprising the endonuclease protein and the guiding RNA.

The method further comprises introducing into the cell or embryos two or more guiding RNAs or DNA encoding two or more guiding RNAs. Guiding RNAs are detailed above in section (IV)(c). Target sites of guiding RNAs are described above in section (IV)(a).

The method can further comprise introducing into the cell or embryo at least one donor polynucleotide comprising at least one sequence having substantial sequence identity with sequence on one side of the targeted site in the chromosomal sequence. Donor polynucleotides are detailed above in section (IV)(e).

The RNA-guided endonucleases (or encoding nucleic acids), guiding RNAs (or encoding DNAs), and the optional donor polynucleotides can be introduced into the cell or embryos using means detailed above in section (IV)(f).

The method further comprises incubating the cell or embryo, as detailed above in section (IV)(g). Suitable cells and embryos are described above in section (IV)(h).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

What is claimed is:

1. A method for modifying a chromosomal sequence in a cell or embryo, the method comprising introducing into the cell or embryo (a) two or more RNA-guided endonucleases or nucleic acid encoding two or more RNA-guided endonucleases and (b) two or more guiding RNAs or DNA encoding two or more guiding RNAs, wherein each guiding RNA guides one of the RNA-guided endonucleases to a targeted site in the chromosomal sequence and the RNA-guided endonuclease cleaves at least one strand of the chromosomal sequence at the targeted site.

2. The method of claim 1, wherein each RNA-guided endonuclease is derived from a Cas9 protein and comprises at least two nuclease domains.

3. The method of claim 2, wherein one of the nuclease domains of each of the two RNA-guided endonucleases is modified such that each RNA-guided endonuclease cleaves one strand of a double-stranded sequence, and wherein the two RNA-guided endonucleases together introduce a double-stranded break in the chromosomal sequence that is repaired by a DNA repair process such that the chromosomal sequence is modified.

4. The method of claim 1, wherein each RNA-guided endonuclease introduces a double-stranded break in the chromosomal sequence that is repaired by a DNA repair process such that the chromosomal sequence is modified.

5. The method of claim 1, further comprising introducing into the cell at least one donor polynucleotide, wherein the donor polynucleotide comprises at least one sequence having substantial sequence identity with sequence on one side of the targeted site in the chromosomal sequence.

6. The method of claim 5, wherein the donor polynucleotide further comprises a donor sequence.

7. The method of claim 6, wherein the donor sequence is integrated into or exchanged with the chromosomal sequence.

8. The method of claim 1, wherein the cell is a human cell, a non-human mammalian cell, a stem cell, a non-mammalian vertebrate cell, an invertebrate cell, a plant cell, or a single cell eukaryotic organism.

9. The method of claim 1, wherein the embryo is a non-human one cell embryo.

10. The method of claim 1, wherein each RNA-guided endonuclease further comprises at least one additional domain chosen from a nuclear localization signal, a cell-penetrating domain, or a marker domain.

11. The method of claim 1, wherein each RNA-guided endonuclease is derived from a Cas9 protein and comprises one functional nuclease domain.

12. The method of claim 11, wherein each RNA-guided endonuclease further comprises at least one nuclear localization signal.


SEQUENCE LISTING (7 sequences)

SEQ ID NO: 1 - protein, 7 aa, Artificial Sequence
  SYNTHESIZED
PKKKRKV

SEQ ID NO: 2 - protein, 7 aa, Artificial Sequence
  SYNTHESIZED
PKKKRRV

SEQ ID NO: 3 - protein, 16 aa, Artificial Sequence
  SYNTHESIZED
KRPAATKKAGQAKKKK

SEQ ID NO: 4 - protein, 20 aa, Artificial Sequence
  SYNTHESIZED
GRKKRRQRRRPPQPKKKRKV

SEQ ID NO: 5 - protein, 19 aa, Artificial Sequence
  SYNTHESIZED
PLSSIFSRIGDPPKKKRKV

SEQ ID NO: 6 - protein, 27 aa, Artificial Sequence
  SYNTHESIZED
GALFLGFLGAAGSTMGAWSQPKKKRKV

SEQ ID NO: 7 - protein, 21 aa, Artificial Sequence
  SYNTHESIZED
KETWWETWWTEWSQPKKKRKV